(19) United States
(12) Plant Patent Application Publication
(10) Pub. No.: US 2003/0121082 P1
(43) Pub. Date: Jun. 26, 2003

(54) Miniature rose plant named 'Benmjul'

(76) Inventor: Frank Benardella, Englishtown, NJ (US)

Correspondence Address: CHRISTIE, PARKER & HALE, LLP, 350 WEST COLORADO BOULEVARD, SUITE 500, PASADENA, CA, 91105, US

(21) Appl. No.: 10/035,940

(22) Filed: Dec. 21, 2001

(51) Int. Cl. A01H 5/00

(52) U.S. Cl. PLT/122

(57) ABSTRACT

A new and distinct Miniature Rose plant having attractive dark red flowers.

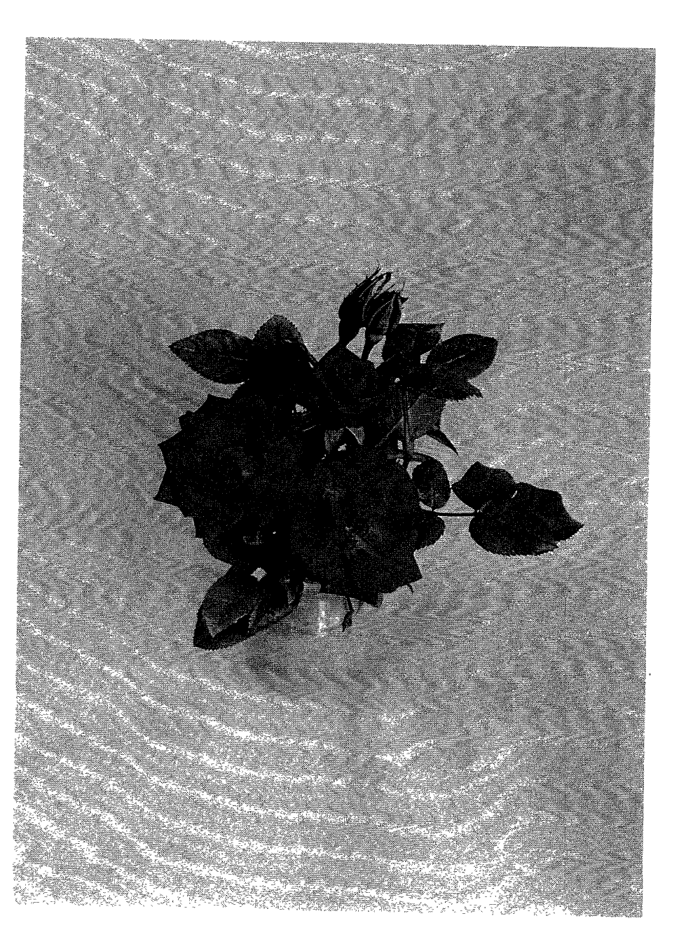

### BACKGROUND OF THE INVENTION

The present invention relates to a new and distinct variety of Miniature rose. The new variety was created by crossing as seed parent the variety known as ‘BENJEN’ (U.S. Plant Pat. No. 5857) and as pollen parent, the variety known as ‘BENMAGIC’ (U.S. Plant Pat. No. 8603). The varietal denomination of the new variety is ‘BENMJUL’.

### SUMMARY OF THE INVENTION

Among the novel characteristics possessed by the new variety which distinguishes it from its parents and all other varieties of which I am aware, is its attractive red flowers.

Asexual reproduction by propagation of vegetative cuttings of the new variety as performed in Essex County, Massachusetts show that the foregoing and other distinguishing characteristics come true to form and are established and transmitted through succeeding propagation.

### COMPARISON WITH PARENTS

The new variety may be distinguished from its parents in flower color.

The new variety has dark red flowers whereas the flowers of its seed parent, ‘BENJEN,’ are light pink.

The flowers of the pollen parent, ‘BENMAGIC,’ are a light red and white bi-color.

### BRIEF DESCRIPTION OF ILLUSTRATION

The accompanying photograph illustrates a typical specimen of the new variety and shows the flowering thereof from bud to full bloom depicted in color as nearly true as it is reasonably possible to make in a color illustration of this character. Throughout this specification, color references and/or values are based upon The Royal Horticultural Society Colour Chart and terminology used in the color descriptions herein refers to plate numbers in this color chart except where common terms of color definition are employed.

### DESCRIPTION OF THE NEW VARIETY

The following observations were made of plants about 3½ years old of the new variety grown outdoors, in 7 inch standard pots, in Essex County, Massachusetts during the month of October. Phenotypic expression may vary with environmental, cultural and climatic conditions, as well as differences in conditions of light and soil.

### FLOWER

Bud.—About ⅝ inch just before sepals divide Form: Obovate Color: When sepals first divide color near 185A; when petals begin to unfurl, color near 53A.

Sepals.—Permanent; extending near ⅜inch beyond tip of bud just before it opens; lanceolate in shape; very finely bearded and with 0 to 4 very fine (narrow) “flags” on each edge of the 3 outer most sepals (never fewer than 1 nor more than 6 on entire sepal) Upper Surface: Texture glabrous; color near 146A, may be flushed lightly with 185A Under surface: Texture very fine down coating; color near 148B.

Hip/Stem Position: Rolling back to stem then coming back to perpendicular with stem as hip forms

Peduncle.—Length: Average about 1 inch Aspect: Straight Strength: Erect Color: Near 144C; may be flushed with near 185A Texture: Some fine pubescence.

### BLOOM

Size.—Average when fully expanded between 1½ to 2 inches.

Borne.—Mostly singly, but also in large sprays.

Form.—High centered at first; becoming and remaining loosely rolled outward

Petalage: Between 21 to 27

Fragrance: Slight; spicy

Persistence: Petals drop off cleanly in 6 to 10 days

Lasting Quality: Between 3 to 6 days as cut flower

### PETALS

Texture: Thick and glabrous

Appearance: Inside velvety; outside satiny

Form: Broad, spatulate but coming to a tip

Size: Length about ⅞ inch at point of attachment to tip; outer petal width about ⅞ inch at widest part

Arrangement: Shingle-like; regularly arranged

Color:

During first 2 days.—Inner Petals: Upper side between 53A and 53B; under side between 184B and 53B Outer Petals: Upper side near 53A; under side between 184A and 53A.

When half-blown.—Inner Petals: Upper side between 46A and 53B; under side between 53D and 57B with an occasional white streak up through center Outer Petals: Upper side between 46A and 53A; under side near 60B.

Base of petals (Point of attachment): Near 1C

General tonality from a distance: Dark red

Color Change: Lightens a little becoming between 53D and 58B and reverse of petals becoming near 58B

General tonality from a distance.—Medium red after 3 or more days.

Petaloids in center: Between 2 to 5

Size.—Average size between ¼ to ⅜ inch wide at widest part; length from point of attachment up to ⅞ inch.

Color.—Same as inner petals.

Texture.—Smooth, velvety inside, satiny outside.

Notable Characteristics: More apt to have white streaks up through the reverse and front

### REPRODUCTIVE ORGANS

Arrangement of stamens, Filaments and Anthers: Arranged around outer edge of receptacle in groups (not bunches) of between 7 to 9 and 3 to 5 singly between each group

Stamens: About 70

Filaments: Length about ¼ inch; color near 58A, translucent, with white tips and at the base

Anthers: Near 17D

Pollen: Some pollen, color near 17B

Pistils: Less than half the quantity of stamens

Styles: Uneven length; between 3/32 to 5/32 inch; color near 180A at upper part and white and translucent toward base

Stigmas: Translucent

Receptacle:

Shape.—Circular; surface texture glabrous.

Size.—Diameter about ¼ inch.

Color.—Near 145D.

Hips:

Shape.—Globular.

Size.—Diameter about 9/16 inch.

Color.—Near 176A.

Seeds: Do not protrude; surface texture glabrous

### PLANT

Form: Upright and well branched

Growth: Vigorous, uniform branched

Size: Height attained between 24 to 30 inches; width about 24 inches

Foliage: Compound of 5 leaflets usually; occasionally 3 or 7 leaflets

Size.—Mature terminal leaflet about 3½ inches from attachment at stem to tip measured along the rachis.

Shape.—Oval pointed; mature terminal leaflet base to tip about 1½ inches.

Quantity.—Abundant.

Color.—New foliage: Upper side between 146A and 137A and margins of near 185A; may be flushed with near 185A in the sun; under side near 146A and edge of near 185A; may be flushed with near 183A Mature foliage: Upper side between 147A and 139A; under side near 147B.

Texture.—Upper side: Semi-glossy Under side: Matte

Edge.—Serrated.

Serration.—Fine and even.

Rachis: Texture glabrous

Color.—Mature Leaf: Upper side color near 144D with edges of between 147A and 139A; under side near 144A Immature Leaf: Upper side near 185A; under side near 182A.

Prickles.—On old foliage; between 2 to 4 on rachis of mature leaves; none observed on main stalks.

Stipules: Finely bearded; length about ½ inch; color on mature leaf near 139D with tips near 139A

Petioles: Texture glabrous

Color.—Upper side on mature leaves color near 144D, under side near 144A.

Resistance to disease: Resistance good against most insects; resistance good against Blackspot and average against Mildew; no resistence to Rust observed

### WOOD

New shoots:

Bark.—Smooth.

Wood.—Near 144B.

Old wood:

Bark.—Smooth and where woody in appearance, texture is somewhat uneven/rough.

Wood.—Near 146C and becoming woody in appearance with slightly raised “strips” of near 199A and near 199C.

Thorns:

Quantity.—Few observed on main stalks from base; many thorns observed on laterals from stalk.

Form.—Slightly angled down to a point; irregularly positioned.

Length.—Medium; about 5/16 inch.

Color.—Immature thorns: Near 166B, but may be between 176A and 178A when in the sun Mature thorns: Near 164B.

Prickles.—No prickles observed on main or lateral stalks; between 2 to 4 prickles on rachis of mature leaves; color near 145C.

Winter Hardiness: Tested hardy to zones 4 to 9; winter protection recommended in Zones 5 and colder

I claim:

1. A new and distinct Miniature rose plant of the variety substantially as shown and described.
